SEQUENCE LISTING (83 sequences)

SEQ ID NO: 1 - DNA, 483 bp, synthetic construct
  source 1..483: mol_type = other DNA
atcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 2 - DNA, 402 bp, synthetic construct
  source 1..402: mol_type = other DNA
aataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 3 - DNA, 483 bp, synthetic construct
  source 1..483: mol_type = other DNA
atcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgac

SEQ ID NO: 4 - DNA, 69 bp, synthetic construct
  source 1..69: mol_type = other DNA
atgggtcgggggctgctcaggggcctgtggccgctgcacatcgtcctgtggacgcgtatcgccagcacg

SEQ ID NO: 5 - DNA, 96 bp, synthetic construct
  source 1..96: mol_type = other DNA
atgcacagtcaagggcggggttgcaacaacacaaaacaaaacaaaacttccggacttcgacctgcagctgagaagaacatctcgcaaagcggcgtt

SEQ ID NO: 6 - DNA, 69 bp, synthetic construct
  source 1..69: mol_type = other DNA
atgggtcgggggctgctccggggcctgtggccgctgcatatcgtcctgtggacgcgcatcgccagcacg

SEQ ID NO: 7 - protein, 23 aa, synthetic construct
MGRGLLRGLWPLHIVLWTRIAST

SEQ ID NO: 8 - protein, 32 aa, synthetic construct
MHSQGRGCNNTKQNKTSGLRPAAEKNISQSGV

SEQ ID NO: 9 - DNA, 552 bp, synthetic construct
  source 1..552: mol_type = other DNA
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 10 - DNA, 498 bp, synthetic construct
  source 1..498: mol_type = other DNA
atgcacagtcaagggcggggttgcaacaacacaaaacaaaacaaaacttccggacttcgacctgcagctgagaagaacatctcgcaaagcggcgttaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 11 - DNA, 471 bp, synthetic construct
  source 1..471: mol_type = other DNA
atgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 12 - DNA, 552 bp, synthetic construct
  source 1..552: mol_type = other DNA
atgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgac

SEQ ID NO: 13 - protein, 161 aa, synthetic construct
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 14 - protein, 161 aa, synthetic construct
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 15 - protein, 127 aa, synthetic construct
NNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPD

SEQ ID NO: 16 - protein, 184 aa, synthetic construct
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 17 - protein, 159 aa, synthetic construct
MHSQGRGCNNTKQNKTSGLRPAAEKNISQSGVNNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPD

SEQ ID NO: 18 - protein, 184 aa, synthetic construct
MGRGLLRGLWPLHIVLWTRIASTIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 19 - protein, 150 aa, synthetic construct
MGRGLLRGLWPLHIVLWTRIASTNNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPD

SEQ ID NO: 20 - DNA, 711 bp, synthetic construct
  source 1..711: mol_type = other DNA
cccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaa

SEQ ID NO: 21 - DNA, 696 bp, synthetic construct
  source 1..696: mol_type = other DNA
cccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 22 - protein, 237 aa, synthetic construct
PKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 23 - protein, 232 aa, synthetic construct
PRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 24 - DNA, 1113 bp, synthetic construct
  source 1..1113: mol_type = other DNA
aataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaa

SEQ ID NO: 25 - DNA, 1182 bp, synthetic construct
  source 1..1182: mol_type = other DNA
atgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaa

SEQ ID NO: 26 - protein, 371 aa, synthetic construct
NNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPDLLLVIFQPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 27 - protein, 394 aa, synthetic construct
MGRGLLRGLWPLHIVLWTRIASTNNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPDLLLVIFQPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 28 - DNA, 627 bp, synthetic construct
  source 1..627: mol_type = other DNA
atggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcc

SEQ ID NO: 29 - DNA, 633 bp, synthetic construct
  source 1..633: mol_type = other DNA
atgggctgggccgaggccgggttcgagcacctgggactgtgggtccctgtgctggctgtgcttttgctggaagcctgccgggcacatccgatccctgactccagccccctcctacaatttggaggtcaagttcgacagcggtacctctacaccgacgatgcccaggagacagaggcccacctagagatcagggccgatggcacagtggtgggggctgcccgccagagccctgaaagtctcctggagctgaaagccctaaagccaggggtcattcaaatcttgggagtcaaaacatccaggttcctgtgccagggcccagatgggacactatatggctcgctccatttcgaccctgtggcctgcagtttccgagaactgcttcttgaggatgggtacaacatctaccactccgagacccttggtctcccgcttcgcctgcgcccccacaactccgcataccgggacttggcaccccgcgggcctgcccgcttcctgccactgccaggcctgcttccagcacccccagagcctccagggatcctggccccggagcctcctgacgtgggctcctcggaccctctgagcatggtggggccttcacagggccggagtcccagctatgcttcctgatag

SEQ ID NO: 30 - DNA, 627 bp, synthetic construct
  source 1..627: mol_type = other DNA
atggggtgggccgaggccgggttcgaacatcttgggctttgggtcccagttctcgctgtcctgcttctggaagcttgccgcgcacatccaatccccgactcttcacccctgctgcagttcggaggacaagtgcgccagagatacctgtacactgatgacgcgcaggaaaccgaggcgcacttggaaattagggcagatgggaccgtggtcggagcagccaggcaaagtcctgagagcctgctcgaattgaaagccctcaagcccggcgtgatccaaatcctgggggttaagacatccagatttctctgccaagggccggatggaaccttgtacggtagccttcatttcgacccagtggcttgttcctttcgagagttgctcctggaagacggatacaatatctaccactccgaaactctcggattgcccttgaggctgagacctcacaactctgcttaccgagacttggctcccaggggtcctgcaagattcctgccgctgccgggattgcttcctgccccaccagagccccctggaatccttgcgccagaaccacccgatgttggctccagtgacccactttcaatggttggcccttcccaaggtagaagtccctcatacgcatcc

SEQ ID NO: 31 - DNA, 633 bp, synthetic construct
  source 1..633: mol_type = other DNA
atggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctga

SEQ ID NO: 32 - protein, 209 aa, synthetic construct
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 33 - protein, 209 aa, synthetic construct
MGWAEAGFEHLGLWVPVLAVLLLEACRAHPIPDSSPLLQFGGQVRQRYLYTDDAQETEAHLEIRADGTVVGAARQSPESLLELKALKPGVIQILGVKTSRFLCQGPDGTLYGSLHFDPVACSFRELLLEDGYNIYHSETLGLPLRLRPHNSAYRDLAPRGPARFLPLPGLLPAPPEPPGILAPEPPDVGSSDPLSMVGPSQGRSPSYAS

SEQ ID NO: 34 - protein, 210 aa, synthetic construct
MEWMRSRVGTLGLWVRLLLAVFLLGVYQAYPIPDSSPLLQFGGQVRQRYLYTDDDQDTEAHLEIREDGTVVGAAHRSPESLLELKALKPGVIQILGVKASRFLCQQPDGALYGSPHFDPEACSFRELLLEDGYNVYQSEAHGLPLRLPQKDSPNQDATSWGPVRFLPMPGLLHEPQDQAGFLPPEPPDVGSSDPLSMVEPLQGRSPSYAS

SEQ ID NO: 35 - DNA, 1153 bp, synthetic construct
  source 1..1153: mol_type = other DNA
gggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggt

SEQ ID NO: 36 - DNA, 231 bp, synthetic construct
  source 1..231: mol_type = other DNA
aggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacaca

SEQ ID NO: 37 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 38 - DNA, 393 bp, synthetic construct
  source 1..393: mol_type = other DNA
atcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtg

SEQ ID NO: 39 - DNA, 154 bp, synthetic construct
  source 1..154: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagc

SEQ ID NO: 40 - DNA, 504 bp, synthetic construct
  source 1..504: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagc

SEQ ID NO: 41 - DNA, 100 bp, synthetic construct
  source 1..100: mol_type = other DNA
ccaaaatcaacgggactttccaaaatgtcgtaataaccccgccccgttgacgcaaatgggcggtaggcgtgtacggtgggaggtctatataagcagagct

SEQ ID NO: 42 - DNA, 1873 bp, synthetic construct
  source 1..1873: mol_type = other DNA
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 43 - DNA, 913 bp, synthetic construct
  source 1..913: mol_type = other DNA
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcacc

SEQ ID NO: 44 - DNA, 1246 bp, synthetic construct
  source 1..1246: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 45 - DNA, 1061 bp, synthetic construct
  source 1..1061: mol_type = other DNA
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 46 - DNA, 953 bp, synthetic construct
  source 1..953: mol_type = other DNA
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 47 - DNA, 82 bp, synthetic construct
  source 1..82: mol_type = other DNA
gtaagggtttaagggatggttggttggtggggtattaatgtttaattacctggagcacctgcctgaaatcactttttttcag

SEQ ID NO: 48 - DNA, 476 bp, synthetic construct
  source 1..476: mol_type = other DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 49 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 50 - DNA, 248 bp, synthetic construct
  source 1..248: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 51 - DNA, 132 bp, synthetic construct
  source 1..132: mol_type = other DNA
gctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 52 - DNA, 510 bp, synthetic construct
  source 1..510: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 53 - DNA, 387 bp, synthetic construct
  source 1..387: mol_type = other DNA
tggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 54 - DNA, 479 bp, synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 55 - DNA, 54 bp, synthetic construct
  source 1..54: mol_type = other DNA
gagggccgcggcagcctgctgacctgcggcgacgtggaggaaaaccccggcccc

SEQ ID NO: 56 - DNA, 59 bp, synthetic construct
  source 1..59: mol_type = other DNA
ggatctggcgccaccaacttctctctgctgaagcaggccggcgacgtggaggagaaccc

SEQ ID NO: 57 - DNA, 581 bp, synthetic construct
  source 1..581: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagccacc

SEQ ID NO: 58 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 59 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 60 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 61 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 62 - DNA, 2214 bp, synthetic construct
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 63 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 64 - protein, 601 aa, synthetic construct
TAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 65 - protein, 535 aa, synthetic construct
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 66 - DNA, 3196 bp, synthetic construct
  source 1..3196: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgagtgatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaataataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 67 - DNA, 4611 bp, synthetic construct
  source 1..4611: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatgaattctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgagtgatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaataataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagccacgcgtgaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggttttttttttttttttgaggtggggaactagataattatggacatctttccatactagcatatcaatatctacctcattctttttaatatttttgctagtattccattgtatgaatgtcctatgatttacttaacctgtccatcaatatttgtttccaggtttttgctattataatgctgctgcaaagtacatcctcacacatctttattttgtctattcatatttctgtaagataggttactaaagttggaactgccaaattaacactatcatactattttgttttttaattttaattttttaaaaaatgtaaaatgtgcaatttcaagaggagaaacttgaacacaaggagcaaaatctatttttataacatcctattaaaagcttgctttacataaagattttgaaagaatagcataaatacatgactagtcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 68 - DNA, 2640 bp, synthetic construct
  source 1..2640: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgaggatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatggggtgggccgaggccgggttcgaacatcttgggctttgggtcccagttctcgctgtcctgcttctggaagcttgccgcgcacatccaatccccgactcttcacccctgctgcagttcggaggacaagtgcgccagagatacctgtacactgatgacgcgcaggaaaccgaggcgcacttggaaattagggcagatgggaccgtggtcggagcagccaggcaaagtcctgagagcctgctcgaattgaaagccctcaagcccggcgtgatccaaatcctgggggttaagacatccagatttctctgccaagggccggatggaaccttgtacggtagccttcatttcgacccagtggcttgttcctttcgagagttgctcctggaagacggatacaatatctaccactccgaaactctcggattgcccttgaggctgagacctcacaactctgcttaccgagacttggctcccaggggtcctgcaagattcctgccgctgccgggattgcttcctgccccaccagagccccctggaatccttgcgccagaaccacccgatgttggctccagtgacccactttcaatggttggcccttcccaaggtagaagtccctcatacgcatcctaataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 69 - DNA, 4596 bp, synthetic construct
  source 1..4596: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctaccggtgaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggttttttttttttttttgaggtggggaactagataattatggacatctttccatactagcatatcaatatctacctcattctttttaatatttttgctagtgttactgtccaatggaattctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgaggatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatggggtgggccgaggccgggttcgaacatcttgggctttgggtcccagttctcgctgtcctgcttctggaagcttgccgcgcacatccaatccccgactcttcacccctgctgcagttcggaggacaagtgcgccagagatacctgtacactgatgacgcgcaggaaaccgaggcgcacttggaaattagggcagatgggaccgtggtcggagcagccaggcaaagtcctgagagcctgctcgaattgaaagccctcaagcccggcgtgatccaaatcctgggggttaagacatccagatttctctgccaagggccggatggaaccttgtacggtagccttcatttcgacccagtggcttgttcctttcgagagttgctcctggaagacggatacaatatctaccactccgaaactctcggattgcccttgaggctgagacctcacaactctgcttaccgagacttggctcccaggggtcctgcaagattcctgccgctgccgggattgcttcctgccccaccagagccccctggaatccttgcgccagaaccacccgatgttggctccagtgacccactttcaatggttggcccttcccaaggtagaagtccctcatacgcatcctaataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagccacgcgtgtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaagtgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcactagtaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 70 - DNA, 235 bp, synthetic construct
  source 1..235: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgaca

SEQ ID NO: 71 - DNA, 1247 bp, synthetic construct
  source 1..1247: mol_type = other DNA
atgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgaccccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaa

SEQ ID NO: 72 - DNA, 1178 bp, synthetic construct
  source 1..1178: mol_type = other DNA
atcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgaccccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaa

SEQ ID NO: 73 - protein, 415 aa, synthetic construct
MGRGLLRGLWPLHIVLWTRIASTIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 74 - protein, 392 aa, synthetic construct
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 75 - DNA, 169 bp, synthetic construct
  source 1..169: mol_type = other DNA
tttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 76 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 77 - DNA, 140 bp, synthetic construct
  source 1..140: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaa

SEQ ID NO: 78 - DNA, 2939 bp, synthetic construct
  source 1..2939: mol_type = other DNA
gggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgcggcctgccaccatgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgaccccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggagggagtggtgggtccgattacaaagatcacgatggggactataaagatcacgacatcgactataaggatgacgatgataaatgaataagctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 79 - DNA, 3379 bp, synthetic construct
  source 1..3379: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattccttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgcggcctgccaccatgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgaccccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaggagggagtggtgggtccgattacaaagatcacgatggggactataaagatcacgacatcgactataaggatgacgatgataaatgaataagctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaa

SEQ ID NO: 80 - DNA, 1319 bp, synthetic construct
  source 1..1319: mol_type = other DNA
aggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagcggccgctcgccaccatggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctgaagcgggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 81 - DNA, 1738 bp, synthetic construct
  source 1..1738: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattccaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagcggccgctcgccaccatggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctgaagcgggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaa

SEQ ID NO: 82 - DNA, 696 bp, synthetic construct; identical to SEQ ID NO: 21
  source 1..696: mol_type = other DNA

SEQ ID NO: 83 - protein, 232 aa, synthetic construct; identical to SEQ ID NO: 23

(19) United States
(12) Patent Application Publication
(10) Pub. No.: US 2026/0325676 A1
(43) Pub. Date: Oct. 22, 2026

(54) GENE THERAPY METHODS FOR FAMILIAL PARTIAL LIPODYSTROPHY

(71) Applicant: Rejuvenate Bio Inc, San Diego, CA (US)

(72) Inventors: Noah Davidsohn, Brookline, MA (US); Dan Oliver, San Diego, CA (US)

(21) Appl. No.: 19/114,106

(22) PCT Filed: Sep. 21, 2023
(86) PCT No.: PCT/US2023/074732

§ 371 Date: Mar. 21, 2025

Related U.S. Application Data
(60) Provisional application No. 63/376,475, filed on Sep. 21, 2022.

(51) Int. Cl. A61K 48/00; A61K 38/18; C12N 15/86

(52) U.S. Cl. CPC A61K 48/005; A61K 38/1825; A61K 38/1841; A61K 48/0075; C12N 15/86; C12N 2750/14143; C12N 2830/48; C12N 2830/50; C12N 2840/203

(57) ABSTRACT

The present disclosure provides methods of treating familial partial lipodystrophy (FPL) in a subject. The methods generally comprise administering to the subject a gene therapy comprising a first nucleic acid encoding soluble transforming growth factor beta receptor 2 (sTGFβR2) and a second nucleic acid encoding fibroblast growth factor 21.

### RELATED APPLICATIONS

This application claims priority to U.S. Provisional Patent Application Ser. No. 63/376,475, filed Sep. 21, 2022, the entire disclosure of which is hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Sep. 19, 2023, is named “203434_SL.xml” and is 131,991 bytes in size).

### BACKGROUND

Familial partial lipodystrophy (FPL) is a rare genetic disorder that results in selective, progressive loss of body fat from various areas of the body. Individuals with FPL typically have reduced subcutaneous fat in the arms and legs, and they may or may not experience loss of body fat in the head and trunk regions. Individuals with FPL may also experience excess subcutaneous fat in other regions of the body, in particular, the neck, face, and intra-abdominal regions. The prevalence of FPL is estimated to be one in a million people; however, many cases may go misdiagnosed or undiagnosed.

The extent of loss of body fat frequently determines the severity of associated metabolic complications. These include an inability to properly break down glucose (i.e., glucose intolerance), elevated levels of triglycerides in the blood (hypertriglyceridemia), and diabetes.

There is no cure for FPL. Treatment of FPL is directed toward the specific symptoms that manifest in each individual, coupled with a specific lifestyle. Individuals with FPL are encouraged to follow a high carbohydrate, low fat diet, which can improve chylomicronemia associated with acute pancreatitis. Regular exercise and maintaining a healthy weight are also encouraged as a way to reduce the risk of developing diabetes. Some individuals may be treated with fibric acid derivatives, statins, or n-3 polyunsaturated fatty acids. In some cases, individuals with FPL that have liver and/or cardiac disease may require transplants.

Accordingly, there is a need in the art for methods for treating familial partial lipodystrophy.

### SUMMARY

The present disclosure provides methods of treating familial partial lipodystrophy (FPL) in a subject. The methods generally comprise administering to the subject a gene therapy comprising a first nucleic acid encoding soluble transforming growth factor beta receptor 2 (sTGFBR2) and a second nucleic acid encoding fibroblast growth factor 21 (FGF21).

Accordingly, in one aspect, the instant disclosure provides a method of treating familial partial lipodystrophy (FPL) in a subject, comprising administering to the subject a gene therapy comprising a first nucleic acid encoding soluble transforming growth factor beta receptor (sTGFBR2) and a second nucleic acid encoding fibroblast growth factor 21 (FGF21).

In certain embodiments, the subject is diagnosed with autosomal recessive FPL, FPL type 1 (Kobberling lipodystrophy), FPL type 2 (Dunnigan lipodystrophy), FPL type 3, FPL type 4, and FPL type 5. In certain embodiments, the subject comprises a genetic mutation in LMNA, PPARG, PLIN1, AKT2, CIDEC, and/or AGPAT2. In certain embodiments, the subject is diagnosed with FPL type 2. In certain embodiments, the subject comprises a genetic mutation in LMNA. In certain embodiments, the subject is a mammal. In certain embodiments, the subject is a human.

In certain embodiments, the method further comprises determining a base level of plasma and/or liver triglycerides in the subject prior to administration of the gene therapy. In certain embodiments, the treatment results in a reduction of the level of plasma and/or liver triglycerides in the subject at a duration after administration of the gene therapy, as compared to the base level of plasma and/or liver triglycerides in the subject.

In certain embodiments, the method further comprises determining a base level of glucose homeostasis in the subject prior to administration of the gene therapy. In certain embodiments, the treatment results in an improvement in glucose homeostasis in the subject at a duration after administration of the gene therapy, as compared to the base level of glucose homeostasis in the subject. In certain embodiments, glucose homeostasis is measured by a glucose tolerance test and/or an insulin tolerance test.

In certain embodiments, the gene therapy is administered intravenously.

In certain embodiments, the first nucleic acid comprises a first transcriptional regulatory element operably linked to the sTGFBR2 coding sequence. In certain embodiments, the sTGFBR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 1, 2, 3, 24, or 72. In certain embodiments, the sTGFBR2 coding sequence further comprises a heterologous or an innate secretion signal sequence, wherein the signal sequence is encoded by a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 4, 5, or 6. In certain embodiments, the sTGFBR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 9, 10, 11, 12, 25, or 71. In certain embodiments, the sTGFBR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 13, 14, 15, 26, or 74. In certain embodiments, the sTGFBR2 coding sequence further encodes a secretion signal sequence having at least 85%, 86%, 87%, 88%, 89%, 90%, 91%, 92%, 93%, 94%, 95%, 96%, 97%, 98%, 99%, or 100% sequence identity to SEQ ID NO: 7 or 8. In certain embodiments, the STGFBR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 16, 17, 18, 19, 27, or 73.

In certain embodiments, the second nucleic acid comprises a second transcriptional regulatory element operably linked to the FGF21 coding sequence. In certain embodiments, the FGF21 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 28, 29, 30, or 31. In certain embodiments, the FGF21 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 32, 33, or 34.

In certain embodiments, the first and second transcriptional regulatory element are each selected from the group consisting of: one or more ApoE binding sites, an hAAT promoter, and an EF-la promoter or variant thereof. In certain embodiments, the first and second transcriptional regulatory element each comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 34, 36, 38, 39, 40, and/or 48.

In certain embodiments, the first and second nucleic acid each further comprises a post-transcriptional regulatory element. In certain embodiments, the post-transcriptional regulatory element comprises a polyadenylation signal and/or WPRE sequence. In certain embodiments, the polyadenylation signal is an SV40 polyadenylation signal. In certain embodiments, the WPRE sequence is a WPRE3 sequence. In certain embodiments, the post-transcriptional regulatory element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 50, 51, 70, or 75.

In certain embodiments, the first nucleic acid is comprised within a first vector, and the second nucleic acid is comprised within a second vector. In certain embodiments, the first vector and/or the second vector is each a viral vector, optionally wherein each is independently selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus. In certain embodiments, the first vector is an AAV vector comprised within a first recombinant AAV (rAAV), wherein the first rAAV comprises an AAV capsid comprising an AAV capsid protein; and a first rAAV genome; and/or the second vector is an AAV vector comprised within a second rAAV, wherein the second rAAV comprises an AAV capsid comprising an AAV capsid protein; and a second rAAV genome.

In certain embodiments, the gene therapy comprises: a first recombinant AAV (rAAV) comprising: an AAV capsid comprising an AAV capsid protein; and a first rAAV genome comprising a nucleic acid encoding the amino acid sequence set forth in SEQ ID NO: 26 or 73; and a second rAAV comprising: an AAV capsid comprising an AAV capsid protein; and a second rAAV genome comprising a nucleic acid encoding the amino acid sequence set forth in SEQ ID NO: 33 or 34.

In certain embodiments, the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 66 or 78. In certain embodiments, the first rAAV genome further comprises a 5′ inverted terminal repeat (5′ ITR) nucleotide sequence, and a 3′ inverted terminal repeat (3′ ITR) nucleotide sequence. In certain embodiments, the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58, 59, or 76, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60, 61, or 77. In certain embodiments, the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 67 or 79.

In certain embodiments, the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 68 or 80. In certain embodiments, the second rAAV genome further comprises a 5′ inverted terminal repeat (5′ ITR) nucleotide sequence, and a 3′ inverted terminal repeat (3′ ITR) nucleotide sequence. In certain embodiments, the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58, 59, or 76, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60, 61, or 77. In certain embodiments, wherein the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 69 or 81.

In certain embodiments, the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof. In certain embodiments, the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.

In certain embodiments, the first and the second nucleic acid are comprised within a vector, optionally wherein the first and the second nucleic acid are separated by a polycistronic element. In certain embodiments, the vector comprises: the first nucleic acid comprising a nucleic acid encoding the amino acid sequence set forth in SEQ ID NO: 26 or 73; and the second nucleic acid comprising a nucleic acid encoding the amino acid sequence set forth in SEQ ID NO: 33 or 34. In certain embodiments, the first and the second nucleic acid are separated by a polycistronic element. In certain embodiments, the polycistronic element is an IRES or 2A sequence. In certain embodiments, the polycistronic element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 55, 56, or 57.

In certain embodiments, the vector is an AAV vector comprised within a recombinant AAV (rAAV), wherein the rAAV comprises an AAV capsid comprising an AAV capsid protein; and a rAAV genome. In certain embodiments, the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof. In certain embodiments, the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.

### DETAILED DESCRIPTION

The present disclosure provides methods of treating familial partial lipodystrophy (FPL) in a subject. The methods generally comprise administering to the subject a gene therapy comprising a first nucleic acid encoding soluble transforming growth factor beta receptor 2 (sTGFBR2) and a second nucleic acid encoding fibroblast growth factor 21 (FGF21).

### Definitions

As used herein, the term “replication-defective adeno-associated virus” refers to an AAV comprising a genome lacking Rep and Cap genes.

As used herein, the term “recombinant AAV genome” or “rAAV genome” refers to a coding sequence operably linked to an exogenous transcriptional regulatory element that mediates expression of the coding sequence when the rAAV genome is introduced into a cell. In certain embodiments, the rAAV genome does not integrate in the chromosomal DNA of the cell. The skilled artisan will appreciate that the portion of a rAAV genome comprising the transcriptional regulatory element operably linked to a coding sequence can be in the sense or antisense orientation relative to direction of transcription of the coding sequence.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Only internal gaps are included in the length, not gaps at the sequence ends.

As used herein, the term “coding sequence” refers to the portion of a complementary DNA (cDNA) that encodes a polypeptide, starting at the start codon and ending at the stop codon. A gene may have one or more coding sequences due to alternative splicing, alternative translation initiation, and variation within the population. A coding sequence may either be wild-type or codon optimized.

As used herein, the term “codon optimized” refers to alteration of a coding sequence of a gene (e.g., by nucleotide substitution) without changing the amino acid sequence of the polypeptide encoded by the coding sequence. Such codon alteration is advantageous in that it may increase the translation efficiency of a coding sequence, and/or prevent recombination with a corresponding sequence of an endogenous gene when a coding sequence is transduced into a cell.

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer sequences. A skilled artisan would appreciate that the promoter and enhancer sequences in a gene may be close in location, and the term “promoter” may refer to a sequence comprising a promoter element and an enhancer sequence. Thus, the term “promoter” does not exclude an enhancer sequence in the sequence. The promoter and enhancer sequences do not need to be derived from the same gene or species, and the sequence of each promoter or enhancer sequence may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer sequences. The coding sequence is “operably linked” to the TRE if the transcription of the coding sequence is controlled or influenced by the TRE. The promoter and enhancer sequences of the TRE may be in any orientation and/or distance from the coding sequence, as long as the desired transcriptional activity is obtained. In certain embodiments, the TRE is upstream from the coding sequence.

As used herein, the term “polyadenylation signal” or “polyadenylation sequence” refers to a DNA sequence that when transcribed into RNA constitutes a polyadenylation signal sequence. The polyadenylation sequence can be native (e.g., with respect to the coding sequence of a gene) or exogenous. The exogenous polyadenylation sequence can be a mammalian or a viral polyadenylation sequence (e.g., an SV40 polyadenylation sequence).

As used herein, “exogenous polyadenylation sequence” refers to a polyadenylation sequence not identical or substantially identical to the endogenous polyadenylation sequence of a coding sequence of a gene. In certain embodiments, an exogenous polyadenylation sequence can be of the same species (e.g., human), or of a different species (e.g., a virus).

As used herein, the term “effective amount” in the context of the administration of a viral vector (e.g., recombinant AAV) to a subject refers to the amount of the viral vector that achieves a desired prophylactic or therapeutic effect. In the context of the administration of a compound, an effective amount refers to the amount of the compound that achieves a desired prophylactic or therapeutic effect.

As used herein, the term “polynucleotide,” in its broadest sense, includes any compound and/or substance that comprise a polymer of nucleotides linked via a phosphodiester bond.

As used herein, the term “treat,” “treating,” and “treatment” refer to therapeutic or preventative measures described herein. The methods of “treatment” employ administration of a polynucleotide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In certain embodiments, the subject is a non-human mammal. In certain embodiments, the subject is a human. In certain embodiments, the subject is a canine.

### Polynucleotides, Vectors, and Compositions

In one aspect, the methods disclosed herein employ a first nucleic acid encoding soluble transforming growth factor beta receptor 2 (sTGFBR2) and a second nucleic acid encoding fibroblast growth factor 21 (FGF21).

In certain embodiments, the sTGFBR2 coding sequence encodes for a polypeptide comprising all or substantially all of the extracellular portion of TGFβR2 and does not encode for any transmembrane or intracellular aspects of TGFβR2. In certain embodiments, the sTGFβR2 coding sequence encodes for a polypeptide comprising the extracellular portion of wild type TGFβR2. In certain embodiments, the sTGFβR2 coding sequence encodes for a polypeptide comprising the extracellular portion of functional variant of TGFβR2. In certain embodiments, the sTGFβR2 coding sequence encodes a human, mouse, or canine TGFβR2.

In certain embodiments, the sTGFβR2 coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 13, 14, or 15. In certain embodiments, the STGFβR2 coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 1, 2, or 3.

In certain embodiments, the sTGFβR2 coding sequence encodes for a polypeptide comprising all or substantially all of the extracellular portion of TGFβR2, wherein the extracellular portion of TGFβR2 comprises a secretion signal sequence. In certain embodiments, the secretion signal sequence is an innate secretion signal sequence. In certain embodiments, the secretion signal sequence is a heterologous secretion signal sequence. The heterologous secretion signal sequence can be from the same protein (e.g., TGFßR2) of a different species, or from a different protein of the same or different species. For example, the heterologous secretion signal sequence can be obtained from a TGFβR2 secretion signal sequence of a different species. Exemplary TGFβR2 secretion signal sequences include, without limitation, the secretion signal sequences from human, mouse, and canine TGFβR2. As such, the sTGFβR2 coding sequence can further encode a heterologous or innate secretion signal sequence. In certain embodiments, the heterologous or innate secretion signal sequence comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 7 or 8. In certain embodiments, the heterologous or innate secretion signal sequence is encoded by a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 4, 5, or 6.

In certain embodiments, the sTGFβR2 coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 16, 17, 18, or 19. In certain embodiments, the sTGFβR2 coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 9, 10, 11 or 12.

In certain embodiments, the sTGFβR2 coding sequence further encodes a peptide to achieve half-life extension. Such peptides include, without limitation, an IgG constant domain or fragment thereof (e.g., the Fc domain), human serum albumin (HSA), or albumin-binding polypeptides. In certain embodiments, the sTGFβR2 coding sequence further encodes an Fc domain (referred to herein as a sTGFβR2-Fc coding sequence). Exemplary Fc domains include wild type Fc domains from human, mouse, or canine IgG1, IgG2, IgG3 or IgG4. In certain embodiments, the Fc domain comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 22, 23, or 83. In certain embodiments, the Fc domain is encoded by a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 20, 21, or 82.

In certain embodiments, the sTGFβR2-Fc coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 26, 27, 73 or 74. In certain embodiments, the sTGFβR2-Fc coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 24, 25, 71 or 72.

In certain embodiments, the FGF21 coding sequence encodes for a wild type FGF21, or a functional variant thereof. In certain embodiments, the FGF21 coding sequence encodes a human, mouse, or canine FGF21. In certain embodiments, the FGF21 coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 32, 33, or 34. In certain embodiments, the FGF21 coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 28, 29, 30, or 31.

In certain embodiments, the first nucleic acid encoding soluble transforming growth factor beta receptor 2 (sTGFβR2) is each operably linked to a first transcriptional regulatory element (TRE). In certain embodiments, the second nucleic acid encoding fibroblast growth factor 21 (FGF21) is operably linked to a second TRE. In certain embodiments, the first TRE and the second TRE are the same. In certain embodiments, the first TRE and the second TRE are different. In certain embodiments, the first TRE and the second TRE comprises one or more common elements. The first TRE and the second TRE can be active in any mammalian cells (e.g., human cells, canine cells).

In certain embodiments, the TRE is active in a broad range of mammalian cells. Such TREs may comprise a constitutive promoter and/or enhancer sequences including a cytomegalovirus (CMV) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 41); a CMV enhancer sequence, a CBA promoter, and the splice acceptor from exon 3 of the rabbit beta-globin gene, collectively called a CAG promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 42); a human calmodulin 1 (CALM1) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 43); a chicken beta actin (CBA) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 44); a CASI promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 45); an smCBA promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 46); a human elongation factor 1 alpha (EF1α) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 35, 36, or 37); an SV40 promoter; a human phosphoglycerate kinase (PGK1) promoter; a human ubiquitin C (Ubc) promoter; a human beta actin promoter; a human neuron-specific enolase (ENO2) promoter; a human beta-glucuronidase (GUSB) promoter; and/or a human Methyl-CpG Binding Protein 2 (MeCP2) promoter. Any of these TREs or elements within these TREs can be combined in any order to drive efficient transcription.

Alternatively, the TRE may be a tissue-specific TRE, i.e., it is active in specific tissue(s) and/or organ(s). A tissue-specific TRE comprises one or more tissue-specific promoter and/or enhancer sequences, and optionally one or more constitutive promoter and/or enhancer sequences. A skilled artisan would appreciate that tissue-specific promoter and/or enhancer sequences can be isolated from genes specifically expressed in the tissue by methods well known in the art.

In certain embodiments, the TRE is liver-specific, i.e., it is active in cells of the liver. Liver-specific TREs include, without limitation, those provided on the Liver Specific Gene Promoter Database (LSPD, rulai.cshl.edu/LSPD/); a human alpha-1-antitrypsin (hAAT) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 38); an apolipoprotein E (ApoE) binding site (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 39 or 40); a human albumin (hAlb) or minimal promoter; a transthyretin (TTR) promoter or TTR-minimal promoter (TTRm); an apolipoprotein A1 (APOA1) promoter or minimal promoter; a complement factor B (CFB) promoter; a ketohexokinase (KHK) promoter; a hemopexin (HPX) promoter or minimal promoter; a nicotinamide N-methyltransferase (NNMT) promoter or minimal promoter; a (liver) carboxylesterase 1 (CES1) promoter or minimal promoter; a protein C (PROC) promoter or minimal promoter; an apolipoprotein C3 (APOC3) promoter or minimal promoter; a mannan-binding lectin serine protease 2 (MASP2) promoter or minimal promoter; a hepcidin antimicrobial peptide (HAMP) promoter or minimal promoter; and a serpin peptidase inhibitor, clade C (antithrombin), member 1 (SERPINC1) promoter or minimal promoter.

The TRE may be an inducible promoter. The use of an inducible promoter provides a molecular switch capable of turning on expression of the polynucleotide sequence which it is operatively linked when such expression is desired, or turning off the expression when expression is not desired. Examples of inducible promoters include, without limitation, a metallothionine promoter, a glucocorticoid promoter, a progesterone promoter, and a tetracycline promoter.

In certain embodiments, the first nucleic acid and/or the second nucleic acid comprises two or more TREs, optionally comprising at least one of the TREs disclosed herein. A skilled person in the art would appreciate that any of these TREs can be combined in any order, and combinations of a constitutive TRE and a tissue-specific TRE can drive efficient and tissue-specific transcription.

In certain embodiments, the TRE can further comprise an intron sequence. Such introns can increase transgene expression, for example, by reducing transcriptional silencing and enhancing mRNA export from the nucleus to the cytoplasm. The intron can comprise a native intron sequence of sTGFβR2 or FGF21, an intron sequence from the same genes of a different species, an intron sequence from a different gene from the same species, and/or a synthetic intron sequence. A skilled worker will appreciate that synthetic intron sequences can be designed to mediate RNA splicing by introducing any consensus splicing motifs known in the art (e.g., in Sibley et al. Nature Reviews Genetics. 2016, 17:407-21, which is incorporated by reference herein in its entirety). Exemplary intron sequences are provided in Lu et al., Molecular Therapy. 2013, 21 (5): 954-63, and Lu et al., Hum. Gene Ther. 2017, 28 (1): 125-34, which are incorporated by reference herein in their entirety. Suitable intron sequence include, without limitation, a minute virus of mouse (MVM) intron (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 47); a β-globin intron sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 48); and an SV40 intron sequence.

In certain embodiments, the first nucleic acid and/or the second nucleic acid further comprises a post-transcriptional regulatory element. The post-transcriptional regulatory element may be any sequence that effectively terminates transcription, and a skilled artisan would appreciate that such sequences can be isolated from any genes that are expressed in the cell in which transcription of the coding sequence is desired.

In certain embodiments, the post-transcriptional regulatory element comprises a polyadenylation signal sequence. In certain embodiments, the polyadenylation signal sequence is identical or substantially identical to the endogenous polyadenylation sequence of the sTGFβR2 or FGF21 gene. In certain embodiments, the polyadenylation signal sequence is an exogenous polyadenylation signal sequence. In certain embodiments, the polyadenylation signal sequence is an SV40 polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 51); a bovine growth hormone polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 52); a rabbit beta globin polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 53); or a human growth hormone polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 54).

In certain embodiments, the post-transcriptional regulatory element comprises a Woodchuck Hepatitis Virus (WHV) post-transcriptional regulatory element (WPRE). In certain embodiments, the post-transcriptional regulatory element comprises a WPRE sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 49); or a WPRE3 sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50 or 70).

The first nucleic acid and/or the second nucleic acid described herein can be transcribed from an expression vector (e.g., a recombinant expression vector). In certain embodiments, the first nucleic acid is comprised within a first vector, and the second nucleic acid is comprised within a second vector. In certain embodiments, the first nucleic acid and the second nucleic acid are comprised within a single vector. Where the first nucleic acid and the second nucleic acid are comprised within a single vector, the first nucleic acid and the second nucleic acid may be separated by a polycistronic element.

In certain embodiments, the polycistronic element comprises a nucleotide sequence that encodes for an internal ribosome entry site (IRES). An IRES is an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunoglobulin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). In certain embodiments, the polycistronic element comprises a nucleotide sequence that encodes for a 2A sequence. A 2A sequence refers to an oligopeptide that allow multiple proteins to be encoded as polyproteins, which dissociate into component proteins upon translation. Various 2A sequences are known to those of skill in the art, including, without limitation, those found in members of the Picornaviridae virus family, e.g., foot-and-mouth disease virus (FMDV), equine rhinitis A virus (ERAVO), Thosea asigna virus (TaV), and porcine tescho virus-1 (PTV-1); and carioviruses such as Theilovirus and encephalomyocarditis viruses. 2A sequences derived from FMDV, ERAV, PTV-1, and TaV are referred to herein as “F2A,” “E2A,” “P2A,” and “T2A,” respectively. In certain embodiments, the polycistronic element comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 55, 56, or 57.

In certain embodiments, the vector is a non-viral vector. Exemplary non-viral vectors include, but are not limited to, plasmid DNA, transposons, episomal plasmids, minicircles, ministrings, and oligonucleotides (e.g., mRNA, naked DNA). In certain embodiments, the non-viral vector is a DNA plasmid vector. In certain embodiments, the non-viral vector is a transposon-based vector. In certain embodiments, the non-viral vector is a Piggy Bac-based vector, or a Sleeping Beauty-based vector.

In certain embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV1, AAV2, AAV3, AAV4, AAV5, AAV6, AAV7, AAV8, AAV9), retrovirus vectors (e.g., MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, MI), flavivirus (e.g., Kunjin, West Nile, Dengue virus), rhabdovirus vectors (e.g., rabies virus, VSV), measles virus vector, Newcastle disease virus vectors, poxvirus vectors, and picornavirus vectors (e.g., Coxsackievirus). In certain embodiments, the viral vector is selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus.

In certain embodiments, the vector is an AAV vector. In certain embodiments, the vector is a single-stranded AAV. In certain embodiments, the vector is a self-complementary AAV.

In certain embodiments, the vector is an AAV vector comprised within a recombinant AAV (rAAV). In certain embodiments, the rAAV comprises an AAV capsid comprising an AAV capsid protein, and a rAAV genome.

A capsid protein from any capsid known the art can be used in the rAAV compositions disclosed herein, including, without limitation, a capsid protein from an AAV1, AAV2, AAV3, AAV4, AAV5, AAV6, AAV7, AAV8, or AAV9 serotype. The capsid protein can be from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof. In certain embodiments, the capsid protein is from AAV8. In certain embodiments, the capsid protein is encoded by a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 62. In certain embodiments, the capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 1-738 of SEQ ID NO: 63; an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 138-738 of SEQ ID NO: 63;

and/or an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 204-738 of SEQ ID NO: 63. In certain embodiments, the capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 63; an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 64; and/or an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 65.

In certain embodiments, the rAAV comprises an AAV capsid comprising an AAV capsid protein, and a rAAV genome comprising a nucleic acid encoding sTGFβR2. In certain embodiments, the rAAV comprises an AAV capsid comprising an AAV capsid protein, and a rAAV genome comprising a nucleic acid encoding sTGFβR2-Fc. In certain embodiments, the rAAV comprises an AAV capsid comprising an AAV capsid protein, and a rAAV genome comprising a nucleic acid encoding an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 26. In certain embodiments, the rAAV comprises an AAV capsid comprising an AAV capsid protein, and a rAAV genome comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 66.

In certain embodiments, the rAAV comprises an AAV capsid comprising an AAV capsid protein, and a rAAV genome comprising a nucleic acid encoding sTGFβR2. In certain embodiments, the rAAV comprises an AAV capsid comprising an AAV capsid protein, and a rAAV genome comprising a nucleic acid encoding sTGFβR2-Fc. In certain embodiments, the rAAV comprises an AAV capsid comprising an AAV capsid protein, and a rAAV genome comprising a nucleic acid encoding an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 73. In certain embodiments, the rAAV comprises an AAV capsid comprising an AAV capsid protein, and a rAAV genome comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 78.

In certain embodiments, the rAAV comprises an AAV capsid comprising an AAV capsid protein, and a rAAV genome comprising a nucleic acid encoding FGF21. In certain embodiments, the rAAV comprises an AAV capsid comprising an AAV capsid protein, and a rAAV genome comprising a nucleic acid encoding an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 33. In certain embodiments, the rAAV comprises an AAV capsid comprising an AAV capsid protein, and a rAAV genome comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 67.

In certain embodiments, the rAAV comprises an AAV capsid comprising an AAV capsid protein, and a rAAV genome comprising a nucleic acid encoding FGF21. In certain embodiments, the rAAV comprises an AAV capsid comprising an AAV capsid protein, and a rAAV genome comprising a nucleic acid encoding an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 34. In certain embodiments, the rAAV comprises an AAV capsid comprising an AAV capsid protein, and a rAAV genome comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 80.

In certain embodiments, the rAAV genome further comprises a 5′ inverted terminal repeat (5′ ITR) nucleotide sequence, and a 3′ inverted terminal repeat (3′ ITR) nucleotide sequence 3′ of the coding sequence. In certain embodiments, the rAAV genome comprises a 5′ ITR 5′ of the TRE, and a 3′ ITR 3′ of the coding sequence. ITR sequences from any AAV serotype or variant thereof can be used in the rAAV genomes disclosed herein. The 5′ and 3′ ITR can be from an AAV of the same serotype or from AAVs of different serotypes.

Exemplary ITRs for use in the rAAV genomes disclosed herein are set forth in SEQ ID NO: 58, 59, 60, 61, 76, and 77.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 58, 59, or 76. In certain embodiments, the 3′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 60, 61, or 77. In certain embodiments, the 5′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 59, and the 3′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 61. In certain embodiments, the 5′ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 76, and the 3′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 77.

In certain embodiments, the rAAV genome comprises from 5′ to 3′: a 5′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 59); a transcriptional regulatory element operably linked to a nucleic acid encoding sTGFβR2 or sTGFβR2-Fc (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 2, 11, 20, 24, 25, 38, 39, 40, 47, and/or 48); a post-transcriptional regulatory element (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50, 51, 70, or 75); and a 3′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 61). In certain embodiments, the rAAV genome comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 67.

In certain embodiments, the rAAV genome comprises from 5′ to 3′: a 5′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 76); a transcriptional regulatory element operably linked to a nucleic acid encoding sTGFβR2 or sTGFβR2-Fc (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 3, 12, 35, 36, 71, and/or 72); a post-transcriptional regulatory element (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50, 51, 70, or 75); and a 3′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 77). In certain embodiments, the rAAV genome comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 79.

In certain embodiments, the rAAV genome comprises from 5′ to 3′: a 5′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 59); a transcriptional regulatory element operably linked to a nucleic acid encoding FGF21 (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 30, 38, 39, 40, 47, and/or 48); a post-transcriptional regulatory element (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50, 51, 70, 75); and a 3′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 61).

In certain embodiments, the rAAV genome comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 69.

In certain embodiments, the rAAV genome comprises from 5′ to 3′: a 5′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 76); a transcriptional regulatory element operably linked to a nucleic acid encoding FGF21 (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 31, 35, and/or 36); a post-transcriptional regulatory element (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50, 51, 70, or 75); and a 3′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 77). In certain embodiments, the rAAV genome comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 81.

In certain embodiments, the rAAV comprises: (a) an AAV capsid protein comprising the amino acid sequence of amino acids 1-738 of SEQ ID NO: 63, the amino acid sequence of amino acids 138-738 of SEQ ID NO: 63, and/or the amino acid sequence of amino acids 204-738 of SEQ ID NO: 63; and (b) an rAAV genome comprising the nucleotide sequence set forth in any one of SEQ ID NOs: 66, 67, 68, or 69. In certain embodiments, the rAAV comprises (a) an AAV capsid protein comprising the amino acid sequence of amino acids 1-738 of SEQ ID NO: 63, and an rAAV genome comprising the nucleotide sequence set forth in any one of SEQ ID NOs: 66, 67, 68, or 69; (b) an AAV capsid protein comprising the amino acid sequence of amino acids 138-738 of SEQ ID NO: 63, and an rAAV genome comprising the nucleotide sequence set forth in any one of SEQ ID NOs: 66, 67, 68, or 69; and/or (c) an AAV capsid protein comprising the amino acid sequence of amino acids 204-738 of SEQ ID NO: 63, and an rAAV genome comprising the nucleotide sequence set forth in any one of SEQ ID NOs: 66, 67, 68, or 69; In certain embodiments, the rAAV comprises: (a) an AAV capsid protein

comprising the amino acid sequence of amino acids 1-738 of SEQ ID NO: 63, the amino acid sequence of amino acids 138-738 of SEQ ID NO: 63, and/or the amino acid sequence of amino acids 204-738 of SEQ ID NO: 63; and (b) an rAAV genome comprising the nucleotide sequence set forth in any one of SEQ ID NOs: 66, 67, 68, or 69. In certain embodiments, the rAAV comprises (a) an AAV capsid protein comprising the amino acid sequence of amino acids 1-738 of SEQ ID NO: 63, and an rAAV genome comprising the nucleotide sequence set forth in any one of SEQ ID NOs: 78, 79, 80, or 81; (b) an AAV capsid protein comprising the amino acid sequence of amino acids 138-738 of SEQ ID NO: 63, and an rAAV genome comprising the nucleotide sequence set forth in any one of SEQ ID NOs: 66, 67, 68, or 69; and/or (c) an AAV capsid protein comprising the amino acid sequence of amino acids 204-738 of SEQ ID NO: 63, and an rAAV genome comprising the nucleotide sequence set forth in any one of SEQ ID NOs: 78, 79, 80, or 81;

In another aspect, the present disclosure provides a polynucleotide comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 66, 67, 68, or 69.

In another aspect, the present disclosure provides a polynucleotide comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 78, 79, 80, or 81.

The polynucleotide can comprise DNA, RNA, modified DNA, modified RNA, or a combination thereof. In certain embodiments, the polynucleotide is an expression vector. In certain embodiments, the polynucleotide is comprised within a viral vector. In certain embodiments, the polynucleotide is comprised within a plasmid vector.

In another aspect, the present disclosure provides pharmaceutical compositions comprising a rAAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., 7th ed., Lippincott, Williams, & Wilkins; and Handbook of Pharmaceutical Excipients (2000) A. H. Kibbe et al., 3rd ed. Amer. Pharmaceutical Assoc.

### Methods of Treatment

Familial partial lipodystrophy (FPL) is a rare genetic disorder that results in selective, progressive loss of body fat from various areas of the body. Individuals with FPL typically have reduced subcutaneous fat in the arms and legs, and they may or may not experience loss of body fat in the head and trunk regions. Individuals with FPL may also experience excess subcutaneous fat in other regions of the body, in particular, the neck, face, and intra-abdominal regions. The prevalence of FPL is estimated to be one in a million people; however, many cases may go misdiagnosed or undiagnosed.

FPL encompasses several subtypes differentiated by the underlying genetic mutation. The specific symptoms, severity, and prognosis can vary greatly between each type of FPL. FPL is caused by mutations of specific genes, and the various subtypes are characterized by the underlying genetic mutation.

The most common form of FPL is FPL type 2 (also known as Dunnigan lipodystrophy), in which affected individuals experience progressive loss of body fat in the arms, legs, and trunk, at around the time of puberty. Fat may accumulate in other areas of the body including the face, neck, and upper back between the shoulder blades. Individuals with FPL experience insulin resistance which may be associated with acanthosis nigricans, hepatomegaly (enlarged liver) in the form of steatosis which may lead to cirrhosis and liver dysfunction, glucose intolerance, hypertriglyceridemia which may result in pancreatitis, diabetes, coronary artery disease and other types of atherosclerotic vascular disease, and muscular dystrophy. Some women with FPL may develop polycystic ovary syndrome. In rare cases, individuals with FPL have a specific mutation in the LMNA gene; such individuals have increased risk of developing cardiomyopathy which can result in congestive heart failure and cardiac arrhythmias.

Other types of FPL include FPL type 1 (also known as Kobberling lidodystrophy), characterized by fat loss that is generally confined to the arms and legs; FPL type 3, caused by a genetic mutation in PPARG, is generally milder than FPL type 2; FPL type 4, caused by a genetic mutation in PLIN1, is characterized by lipodystrophy most prominent in the lower limbs and buttocks; FPL type 5, caused by a genetic mutation in AKT2, is characterized by lipodystrophy most prominently affecting arms and legs; and autosomal recessive FPL (also known as FPL type 6), caused by a genetic mutation in CIDEC. FPL has also been associated with a genetic mutation in AGPAT2.

Diagnosis of FPL is based upon the identification of characteristic symptoms coupled with clinical testing, and molecular genetic testing to detect mutations in genes that cause FPL.

In another aspect, the present disclosure provides methods for treating familial partial lipodystrophy (FPL) in a subject. The methods generally comprise administering to the subject an effective amount of a gene therapy comprising a first nucleic acid encoding soluble transforming growth factor beta receptor 2 (sTGFβR2) and a second nucleic acid encoding fibroblast growth factor 21 (FGF21).

In certain embodiments, a method for treating FPL in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating FPL in a subject comprises administering: (a) a first rAAV comprising an AAV capsid comprising an AAV capsid protein, and a first rAAV genome comprising a nucleic acid encoding the amino acid sequence set forth in SEQ ID NO: 26; and (b) a second rAAV comprising an AAV capsid comprising an AAV capsid protein, and a second rAAV genome comprising a nucleic acid encoding the amino acid sequence set forth in SEQ ID NO: 33. In certain embodiments, a method for treating FPL in a subject comprises administering: (a) a first rAAV comprising an AAV capsid comprising an AAV capsid protein, and a first rAAV genome comprising the nucleotide sequence set forth in SEQ ID NO: 66 or 68; and (b) a second rAAV comprising an AAV capsid comprising an AAV capsid protein, and a second rAAV genome comprising the nucleotide sequence set forth in SEQ ID NO: 67 or 69.

In certain embodiments, a method for treating FPL in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating FPL in a subject comprises administering: (a) a first rAAV comprising an AAV capsid comprising an AAV capsid protein, and a first rAAV genome comprising a nucleic acid encoding the amino acid sequence set forth in SEQ ID NO: 73; and (b) a second rAAV comprising an AAV capsid comprising an AAV capsid protein, and a second rAAV genome comprising a nucleic acid encoding the amino acid sequence set forth in SEQ ID NO: 74. In certain embodiments, a method for treating FPL in a subject comprises administering: (a) a first rAAV comprising an AAV capsid comprising an AAV capsid protein, and a first rAAV genome comprising the nucleotide sequence set forth in SEQ ID NO: 78 or 79; and (b) a second rAAV comprising an AAV capsid comprising an AAV capsid protein, and a second rAAV genome comprising the nucleotide sequence set forth in SEQ ID NO: 80 or 81.

In certain embodiments, a method for treating FPL in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating FPL in a subject comprises administering an rAAV comprising an AAV capsid comprising an AAV capsid protein, and an rAAV genome comprising: a first nucleic acid encoding the amino acid sequence set forth in SEQ ID NO: 26 and a second nucleic acid encoding the amino acid sequence set forth in SEQ ID NO: 33, wherein the first nucleic acid and the second nucleic acid are separated by a polycistronic element.

In certain embodiments, a method for treating FPL in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating FPL in a subject comprises administering an rAAV comprising an AAV capsid comprising an AAV capsid protein, and an rAAV genome comprising: a first nucleic acid encoding the amino acid sequence set forth in SEQ ID NO: 73 and a second nucleic acid encoding the amino acid sequence set forth in SEQ ID NO: 34, wherein the first nucleic acid and the second nucleic acid are separated by a polycistronic element.

In certain embodiments, a method for treating FPL in a subject further comprises administering to the subject an effective amount of one or more additional therapeutics to treat FPL (e.g., a non-gene therapy therapeutic to treat FPL). Such additional therapeutics to treat FPL may include, without limitation, fibric acid and derivatives thereof, statins, n-3 polyunsaturated fatty acids, hyperglycemic drugs such as metformin and sulfonylureas, insulin, anti-hypertensives, and/or metreleptin.

In certain embodiments, a method for treating FPL in a subject comprises administering to the subject: (a) an effective amount of a gene therapy comprising a first nucleic acid encoding soluble transforming growth factor beta receptor 2 (sTGFβR2) and a second nucleic acid encoding fibroblast growth factor 21 (FGF21); and (b) an effective amount of one or more additional therapeutics to treat FPL. In certain embodiments, a method for treating FPL in a subject comprises administering to the subject: (a) a first rAAV comprising an AAV capsid comprising an AAV capsid protein and a first rAAV genome comprising a nucleic acid encoding sTGFβR2, and a second rAAV comprising an AAV capsid comprising an AAV capsid protein and a second rAAV genome comprising a nucleic acid encoding FGF21; and (b) an effective amount of one or more additional therapeutics to treat FPL. In certain embodiments, a method for treating FPL in a subject comprises administering to the subject: (a) an rAAV comprising an AAV capsid comprising an AAV capsid protein, and an rAAV genome comprising: a first nucleic acid encoding sTGFβR2 and a second nucleic acid encoding

FGF21, wherein the first nucleic acid and the second nucleic acid are separated by a polycistronic element; and (b) an effective amount of one or more additional therapeutics to treat FPL.

In certain embodiments, the one or more additional therapeutics to treat FPL is administered at the same time as the gene therapy. In certain embodiments, the one or more additional therapeutics to treat FPL is administered at a different time as the gene therapy.

In certain embodiments, a method of treating FPL in a subject comprises administering to a subject that has received an effective amount of a non-gene therapy therapeutic to treat FPL, an effective amount of a gene therapy comprising a first nucleic acid encoding soluble transforming growth factor beta receptor 2 (sTGFβR2) and a second nucleic acid encoding fibroblast growth factor 21 (FGF21). In certain embodiments, a method of treating FPL in a subject comprises administering to a subject that has received an effective amount of a non-gene therapy therapeutic to treat FPL, an effective amount of a gene therapy comprising a first nucleic acid encoding soluble transforming growth factor beta receptor 2 (sTGFβR2) and a second nucleic acid encoding fibroblast growth factor 21 (FGF21).

In certain embodiments, the subject is diagnosed with autosomal recessive FPL (also known as FPL type 6), FPL type 1 (also known as Kobberling lipodystrophy), FPL type 2 (also known as Dunnigan lipodystrophy), FPL type 3, FPL type 4, or FPL type 5. In certain embodiments, the subject comprises a genetic mutation in LMNA, PPARG, PLIN1, AKT2, CIDEC, and/or AGPAT2. In certain embodiments, the subject is diagnosed with FPL type 2 and/or comprises a genetic mutation in LMNA. In certain embodiments, the subject is diagnosed with FPL type 2 and comprises a genetic mutation in LMNA.

In certain embodiments, the gene therapy is administered to the subject intravenously, intraperitoneally, subcutaneously, intramuscularly, intrathecally, or intradermally.

In certain embodiments, the subject is a member of any mammalian or non-mammalian species. Suitable subjects include, without limitation, humans, non-human primates, canines, felines, ungulates (e.g., equine, bovine, swine (e.g., pig)), avians, rodents (e.g., rats, mice), and other subjects. In certain embodiments, the subject is human. In certain embodiments, the subject is canine.

### EXAMPLES

The following examples are offered by way of illustration, and not by way of limitation.

### Example 1: STGFβR2 and FGF21 Recombinant AAV Vectors

This example provides sTGFβR2 and FGF21 recombinant adeno-associated virus (rAAV) vectors for expression of sTGFβR2 and FGF21 in a cell (e.g., a liver cell) to which the vectors are transduced.

rAAV-sTGFβR2 comprises a rAAV genome comprising from 5′ to 3′ the following genetic elements: a 5′ ITR element, an EF-la promoter, a sTGFβR2-Fc coding sequence, a WPRE3 sequence, an SV40 polyadenylation signal, and a 3′ ITR element. The sequences of these elements are set forth in Table 1. This vector is capable of expressing a STGFβR2-Fc fusion protein in a cell (e.g., a liver cell) to which the vector is transduced.

rAAV-FGF21 comprises a rAAV genome comprising from 5′ to 3′ the following genetic elements: a 5′ ITR element, an EF-la promoter, a FGF21 coding sequence, a WPRE3 sequence, an SV40 polyadenylation signal, and a 3′ ITR element. The sequences of these elements are set forth in Table 1. This vector is capable of expressing a FGF21 protein in a cell (e.g., a liver cell) to which the vector is transduced.

TABLE 1
Genetic Elements in rAAV-sTGFβR2 and rAAV-FGF21

| | rAAV-sTGFβR2 | rAAV-FGF21 |
|---|---|---|
| Genetic Element | SEQ ID NO: | |
| 5′ ITR element | 76 | 76 |
| EF-1α promoter sequence | 35 | 36 |
| Coding sequence | 71 | 31 |
| Encoded sequence | 73 | 34 |
| WPRE3 sequence | 50 | 50 |
| SV40 polyadenylation signal | 75 | 75 |
| 3′ ITR element | 77 | 77 |
| rAAV genome (promoter to | 78 | 80 |
| polyadenylation signal) | | |
| rAAV genome | 79 | 81 |
| (5′ ITR to 3′ ITR) | | |

The rAAV vectors disclosed herein can be packaged in an AAV capsid, such as, without limitation, an AAV8 capsid. In general, viral particles were generated using standard triple transfection of HEK293T cells and iodixanol gradient purification. See, e.g., Davidsohn et al. (2019) Proc. Natl. Acad. Sci. 116 (47): 23505-23511. The packaged viral particles can be administered to a wild-type animal, or a subject suffering from familial partial lipodystrophy.

### Example 2: Effect of Environmental Temperature on Therapeutic Response

To investigate the effect of environmental temperature on the efficacy of STGFβR2 and FGF21 gene therapy, male and female C57BI/6J mice are administered, via single tail vein intravenous injection, rAAV-sTGFβR2 and rAAV-FGF21, each packaged in AAV8 capsid (AAV8-sTGFβR2 and AAV8-FGF21, respectively). AAV8-sTGFβR2 is administered at a dose of 3E12 vg/kg, and AAV8-FGF21 is administered at dose of 3E12 vg/kg.

Mice are housed at 22° C., 26° C., or 30° C. (thermoneutrality) and fed a high fat diet (HFD; 60% of calories obtained from fat) until 16 weeks of age. At 14 weeks of age, telemeters are implanted into the peritoneal cavities of each animal for measurements of core body temperature. Baseline measurements of metabolism, body composition and core temperatures are made. AAV8-sTGFβR2 and AAV8-FGF21 are then administered, and mice kept on the HFD for an additional four to eight weeks, depending on efficacy of therapy, as assessed by reductions in circulating triglycerides. At sacrifice, organs and blood are collected and processed for further analyses. Two weeks prior to sacrifice, a glucose tolerance test is performed, and a few days prior to sacrifice, an insulin tolerance test is performed.

The following variables are measured: body weight, body composition (by nuclear magnetic resonance), organ weights, core body temperature, liver triglycerides, plasma triglycerides, glucose concentrations, insulin concentrations, and levels of sTGFβR2 and FGF21.

It is expected that animals administered AAV8-sTGFβR2 and AAV8-FGF21 will result in reductions of plasma and liver triglycerides, and improvements in glucose homeostasis, compared to control animals.

### Example 3: Effect of Therapy on Metabolic Dysfunction of Lipodystrophic Mice Fed a High Fat Diet

To investigate the effect of AAV8-sTGFβR2 and AAV8-FGF21 gene therapy on metabolic dysfunction of lipodystrophic mice, four to six constitutive adiponectin-CRE+Lmna^{fl/fl} mice (See, e.g., Corsa et al. (2021) Diabetes 70 (9): 1970-1984) per group are administered, via single intravenous injection, AAV8-sTGFβR2 and AAV8-FGF21. AAV8-sTGFβR2 is administered at a dose of 3E12 vg/kg, and AAV8-FGF21 is administered at dose of 3E11 vg/kg.

To develop liver steatosis and metabolic dysfunction, lipodystrophic animals at about 12 weeks of age are fed a HFD for four weeks. Animals are housed at an environmental temperature determined in Example 1. Baseline measurements of metabolism and body composition are made. AAV8-sTGFβR2 and AAV8-FGF21 is then administered, and mice kept on the HFD for an additional four to eight weeks, at which point they will be sacrificed. Organs and blood are collected and processed for further analyses. Two weeks prior to sacrifice, a glucose tolerance test is performed, and a few days prior to sacrifice, an insulin tolerance test is performed.

The following variables are measured: body weights, body composition, organ weights, liver triglycerides, plasma triglycerides, blood glucose, insulin concentrations, levels of sTGFβR2 and FGF21, circulating markers of liver health, liver histology, targeted liver mRNAs, food intake (crudely), and bone variables.

It is expected that animals administered AAV8-sTGFβR2 and AAV8-FGF21 will result in reductions of plasma and liver triglycerides, and improvements in glucose homeostasis, compared to control animals.

### Example 4: Effect of Therapy on Metabolic Dysfunction of Lipodystrophic Mice Fed a High Fat Diet

To investigate the effect of AAV8-sTGFβR2 and AAV8-FGF21 gene therapy on metabolic dysfunction of lipodystrophic mice, ten to twelve constitutive adiponectin-CRE+Lmna^{fl/fl} mice (See, e.g., Corsa et al. (2021) Diabetes 70 (9): 1970-1984) per group are administered, via single intravenous injection, AAV8-sTGFβR2 and AAV8-FGF21. AAV8-sTGFβR2 is administered at a dose of 3E12 vg/kg, and AAV8-FGF21 is administered at dose of 3E11 vg/kg.

To develop liver steatosis and metabolic dysfunction, lipodystrophic animals at about 12 weeks of age are fed a HFD for four weeks. Animals are housed at an environmental temperature determined in Example 1. Baseline measurements of metabolism and body composition are made. AAV8-sTGFβR2 and AAV8-FGF21 is then administered, and mice kept on the HFD for an additional four to eight weeks, at which point they will be sacrificed. Organs and blood are collected and processed for further analyses. Two weeks prior to sacrifice, a glucose tolerance test is performed, and a few days prior to sacrifice, an insulin tolerance test is performed. CLAMS analyses (Comprehensive Lab Animal Monitoring System: the incorporation of sub-systems for open circuit calorimetry, activity, feeding, running wheel, and sleep detection in an optional environmental chamber) is performed on a subset of mice (16 total) to measure energy expenditure.

The following variables are measured: body weights, body composition (by magnetic resonance imaging), organ weights, liver triglycerides, plasma triglycerides, glucose concentrations, insulin concentrations, levels of sTGFβR2 and FGF21, circulating markers of liver health, liver histology, targeted liver mRNAs (via global RNA sequencing), food intake (crudely), and bone variables.

It is expected that animals administered AAV8-sTGFβR2 and AAV8-FGF21 will result in reductions of plasma and liver triglycerides, and improvements in glucose homeostasis, compared to control animals.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

We claim:

1. A method of treating familial partial lipodystrophy (FPL) in a subject, comprising administering to the subject a gene therapy comprising a first nucleic acid encoding soluble transforming growth factor beta receptor (sTGFβR2) and a second nucleic acid encoding fibroblast growth factor 21 (FGF21).

2. The method of claim 1, wherein the subject is diagnosed with autosomal recessive FPL, FPL type 1 (Kobberling lipodystrophy), FPL type 2 (Dunnigan lipodystrophy), FPL type 3, FPL type 4, and FPL type 5.

3. The method of claim 1 or 2, wherein the subject comprises a genetic mutation in LMNA, PPARG, PLIN1, AKT2, CIDEC, and/or AGPAT2.

4. The method of any one of claims 1-3, wherein the subject is diagnosed with FPL type 2.

5. The method of claim 4, wherein the subject comprises a genetic mutation in LMNA.

6. The method of any one of claims 1-5, wherein the subject is a mammal.

7. The method of any one of claims 1-6, wherein the subject is a human.

8. The method of any one of claims 1-7, further comprising determining a base level of plasma and/or liver triglycerides in the subject prior to administration of the gene therapy.

9. The method of claim 8, wherein the treatment results in a reduction of the level of plasma and/or liver triglycerides in the subject at a duration after administration of the gene therapy, as compared to the base level of plasma and/or liver triglycerides in the subject.

10. The method of any one of claims 1-9, further comprising determining a base level of glucose homeostasis in the subject prior to administration of the gene therapy.

11. The method of claim 10, wherein the treatment results in an improvement in glucose homeostasis in the subject at a duration after administration of the gene therapy, as compared to the base level of glucose homeostasis in the subject.

12. The method of claim 10 or 11, wherein glucose homeostasis is measured by a glucose tolerance test and/or an insulin tolerance test.

13. The method of any one of claims 1-12, wherein the gene therapy is administered intravenously.

14. The method of any one of claims 1-13, wherein the first nucleic acid comprises a first transcriptional regulatory element operably linked to the sTGFβR2 coding sequence.

15. The method of any one of claims 1-14, wherein the sTGFβR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 1, 2, 3, 24, or 72.

16. The method any one of claims 1-15, wherein the sTGFβR2 coding sequence further comprises a heterologous or an innate secretion signal sequence, wherein the signal sequence is encoded by a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 4, 5, or 6.

17. The method of any one of claims 1-16, wherein the sTGFβR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 9, 10, 11, 12, 25, or 71.

18. The method of any one of claims 1-17, wherein the sTGFβR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 13, 14, 15, 26, or 74.

19. The method of claim 18, wherein the sTGFβR2 coding sequence further encodes a secretion signal sequence having at least 85%, 86%, 87%, 88%, 89%, 90%, 91%, 92%, 93%, 94%, 95%, 96%, 97%, 98%, 99%, or 100% sequence identity to SEQ ID NO: 7 or 8.

20. The method of claim 18 or 19, wherein the sTGFβR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 16, 17, 18, 19, 27, or 73.

21. The method of any one of claims 1-20 wherein the second nucleic acid comprises a second transcriptional regulatory element operably linked to the FGF21 coding sequence.

22. The method of any one of claims 1-21, wherein the FGF21 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 28, 29, 30, or 31.

23. The method any one of claims 1-22, wherein the FGF21 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 32, 33, or 34.

24. The method of any one of claims 21-23, wherein the first and second transcriptional regulatory element are each selected from the group consisting of: one or more ApoE binding sites, an hAAT promoter, and an EF-la promoter or variant thereof.

25. The method of any one of claims 21-24, wherein the first and second transcriptional regulatory element each comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 34, 36, 38, 39, 40, and/or 48.

26. The method of any one of claims 1-25, wherein the first and second nucleic acid each further comprises a post-transcriptional regulatory element.

27. The method of claim 26, wherein the post-transcriptional regulatory element comprises a polyadenylation signal and/or WPRE sequence.

28. The method of claim 27, wherein the polyadenylation signal is an SV40 polyadenylation signal.

29. The method of claim 27, wherein the WPRE sequence is a WPRE3 sequence.

30. The method of any one of claims 26-29, wherein the post-transcriptional regulatory element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 50, 51, 70, or 75.

31. The method of any one of claims 1-30, wherein the first nucleic acid is comprised within a first vector, and the second nucleic acid is comprised within a second vector.

32. The method of claim 31, wherein the first vector and/or the second vector is each a viral vector, optionally wherein each is independently selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus.

33. The method of claim 31 or 32, wherein:
the first vector is an AAV vector comprised within a first recombinant AAV (rAAV), wherein the first rAAV comprises an AAV capsid comprising an AAV capsid protein; and a first rAAV genome; and/or
the second vector is an AAV vector comprised within a second rAAV, wherein the second rAAV comprises an AAV capsid comprising an AAV capsid protein; and a second rAAV genome.

34. The method of any one of claims 1-33, wherein the gene therapy comprises:
a first recombinant AAV (rAAV) comprising:
an AAV capsid comprising an AAV capsid protein; and
a first rAAV genome comprising a nucleic acid encoding the amino acid sequence set forth in SEQ ID NO: 26 or 73; and
a second rAAV comprising:
an AAV capsid comprising an AAV capsid protein; and
a second rAAV genome comprising a nucleic acid encoding the amino acid sequence set forth in SEQ ID NO: 33 or 34.

35. The method of claim 33 or 34, wherein the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 66 or 78.

36. The method of any one of claims 33-35, wherein the first rAAV genome further comprises a 5′ inverted terminal repeat (5′ ITR) nucleotide sequence, and a 3′ inverted terminal repeat (3′ ITR) nucleotide sequence.

37. The method of any one of claims 33-36, wherein the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58, 59, or 76, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60, 61, or 77.

38. The method of any one of claims 33-37, wherein the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 67 or 79.

39. The method of any one of claims 33-38, wherein the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 68 or 80.

40. The method of any one of claims 33-39, wherein the second rAAV genome further comprises a 5′ inverted terminal repeat (5′ ITR) nucleotide sequence, and a 3′ inverted terminal repeat (3′ ITR) nucleotide sequence.

41. The method of claim 40, wherein the 5′ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58, 59, or 76, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60, 61, or 77.

42. The method of any one of claims 33-41, wherein the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 69 or 81.

43. The method of any one of claims 33-42, wherein the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

44. The method of any one of claims 33-43, wherein the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.

45. The method of any one of claims 1-30, wherein the first and the second nucleic acid are comprised within a vector, optionally wherein the first and the second nucleic acid are separated by a polycistronic element.

46. The method of claim 45, wherein the vector comprises:
the first nucleic acid comprising a nucleic acid encoding the amino acid sequence set forth in SEQ ID NO: 26 or 73; and
the second nucleic acid comprising a nucleic acid encoding the amino acid sequence set forth in SEQ ID NO: 33 or 34.

47. The method of claim 45 or 46, wherein the first and the second nucleic acid are separated by a polycistronic element.

48. The method of claim 47, wherein the polycistronic element is an IRES or 2A sequence.

49. The method of claim 47 or 48, wherein the polycistronic element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 55, 56, or 57.

50. The method of any one of claims 45-49, wherein the vector is an AAV vector comprised within a recombinant AAV (rAAV), wherein the rAAV comprises an AAV capsid comprising an AAV capsid protein; and a rAAV genome.

51. The method of claim 50, wherein the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

52. The method of claim 50 or 51, wherein the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.
